(19) United States
(12) Patent Application Publication
(10) Pub. No.: US 2006/0173516 A1
(43) Pub. Date: Aug. 3, 2006

(54) Tanning shower-bath booth

(76) Inventor: Candido Bruto, MILANO (IT)

Correspondence Address: AKERMAN SENTERFITT, P.O. BOX 3188, WEST PALM BEACH, FL, 33402-3188, US

(21) Appl. No.: 10/545,919

(22) PCT Filed: Feb. 17, 2004
(86) PCT No.: PCT/EP04/01496

§ 371 Date: Feb. 16, 2006

(30) Foreign Application Priority Data
Feb. 17, 2003 (IT) MI2003A000285

(51) Int. Cl. A61N 5/06

(52) U.S. Cl. 607/94

(57) ABSTRACT

The invention relates to a shower-bath booth, comprising a shower booth having at least a transparent wall and an UV radiation source associated with the shower booth. Advantageously according an embodiment of to the invention, the shower-bath booth comprises a panel, structurally independent from the shower booth and equipped with an UV radiation source, means being provided to apply the panel, in a prefixed spaced relation, to the transparent wall.

### FIELD OF THE INVENTION

The present invention relates, in general, to the technical field of the sanitary fixture industry and, more particularly, it refers to a shower-bath booth of the type comprising tanning lamps.

### BACKGROUND OF THE INVENTION

Apparatuses capable of emitting ultraviolet radiation or UV-A radiation's, used for tanning skin mainly in beauty centres are known since long.

The diffusion of these apparatuses has not reached, since now, if not at a minimum extent, private residences, due to either the high cost and, above all, the considerable space needed to provide a proper installation thereof.

In order to overcome this limitation, it has been thought to associate the “tanning” function with one or more traditionally functions being already present in the domestic environment; for example and in particular the desired “tanning” function has been associated with or attributed to the shower room.

So, shower-bath booths equipped with “tanning” lamps have been realised and put on the market, which allow an exposition to ultraviolet light possibly also during the time spent to have a shower.

Similar shower-bath booths are described in the patent application n. WO 02/47526 filed on Nov. 12, 2001 in the name of the same Applicant.

Despite the advantages of the above shower-bath booths, their diffusion is still discouraged since they are structurally complex and thus expensive.

Moreover, these shower-bath booths suffer from some other drawbacks such as little use elasticity in relation with the different needs, essentially due to their poor if not at all present manoeuvrability.

### SUMMARY OF THE INVENTION

An embodiment of the present invention is directed to a shower-bath booth, which allows the user to get “sun” tanned possibly also while having a shower, having such structural characteristics as to overcome the above drawbacks, i.e. such as to be structurally simple, thus allowing a low realisation cost, and such as to have a use versatility and a manoeuvrability being higher than that of the apparatuses available up to now.

The shower-bath booth comprises a shower booth having at least a transparent wall, an UV radiation source associated with the booth, which comprises a panel, structurally independent from the shower booth and equipped with the UV radiation source, means being provided to apply the panel, in a prefixed spaced relation, to the transparent wall.

Further characteristics and advantages of the present invention will become apparent from the following description of an embodiment thereof given by way of indicative and non limiting example, with reference to the attached drawings.

### BRIEF DESCRIPTION OF THE DRAWINGS

FIG. 1 is a perspective view of a tanning shower-bath booth according to the present invention;

FIG. 2A and 2B are respective front and side views of a detail of the tanning shower-bath booth of FIG. 1;

FIG. 3 schematically shows a further detail of the tanning shower-bath booth of FIG. 1;

FIG. 4 schematically shows a further detail of the tanning shower-bath booth of FIG. 1, according to a further embodiment of the invention;

FIG. 5 schematically shows a longitudinal section view of a detail of the tanning shower-bath booth of FIG. 1.

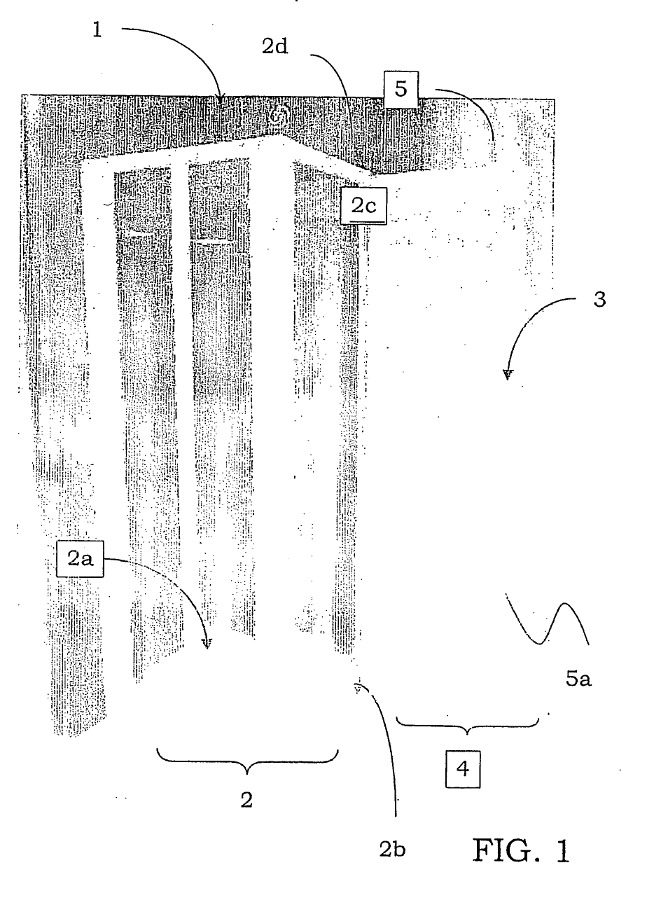

### DETAILED DESCRIPTION

With reference to the above Figures, 1 generally indicates a tanning shower-bath booth realized according to the present invention. The shower-bath booth 1 essentially comprises a shower booth 2, known per se, and an UV radiation source generally indicated with 3.

2A indicates the shallow basin of the shower booth 2, having a conventionally shaped profile 2b, whereas 2c indicates a wall of the shower booth 2, which wall comprises a structure frame globally indicated with 2d.

According to an embodiment of the present invention, the UV radiation source 3 is associated with a panel 4, structurally independent from the shower booth 2, destined to be applied to the side wall 2c of the shower booth 2.

Advantageously, the panel 4 is a box-shaped body 5, defining a space 5a, wherein, according to a preferred embodiment, the UV radiation source 3 is housed.

The box-shaped body 5 is essentially in the form of a rectangular section parallelepiped, wherein an intermediate portion 6 comprised between end portions 7, 8 is located.

The box-shaped body 5 also comprises a wall 9 closing the space 5a and protecting the UV radiation source 3.

In correspondence with the intermediate portion 6, the wall 9 is realised in transparent material, such as glass or, preferably plastic material, e.g. plastic material commercially known as Perspex.

In the space 5a of the box-shaped body 5 and in correspondence with this intermediate portion 6, the UV radiation source 3 is mounted.

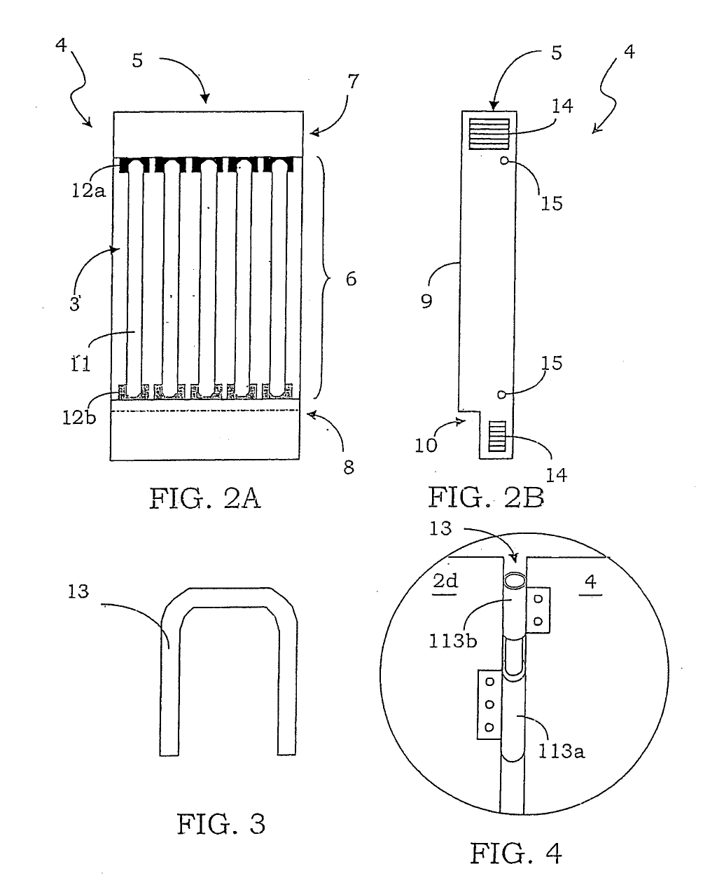

Preferably the source 3 comprises a plurality of UV light tubular lamps 11, mounted on respective holders 12a and 12b and longitudinally extended inside the box-shaped body 5, as schematically shown in FIG. 2A, which shows a front view of the panel 4.

In one of the end portions 7, 8 the box-shaped body 5 has, on the wall 9, a step-like recess 10, extended transversally to the wall 9, as schematically shown in FIG. 2B, which shows a side view of the panel 4.

The step-like recess 10 has a profile conjugated with the profile 2b of the shallow basin 2a edge.

Advantageously, according to an embodiment of the present invention, the connections and the electric circuits, besides the electronic devices controlling the lamps 11, not shown because known per se, are housed inside the box-shaped body 5 in correspondence with the end portions 7,8.

Advantageously, according to an embodiment of the present invention, the shower-bath booth 1 comprises means 13 allowing the application of the panel 4 to the frame 2d of the wall 2c of the shower booth 2.

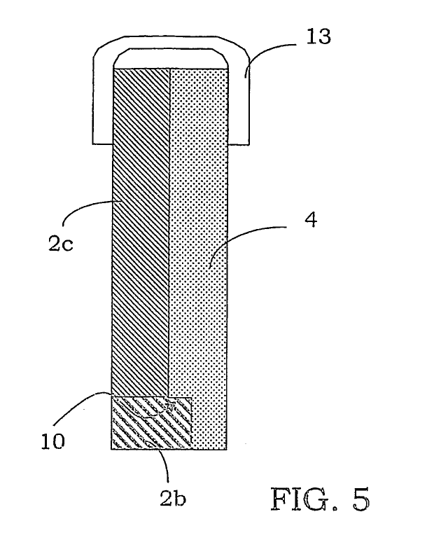

In this way the panel 4, applied by means of the application means 13 and thanks to the step-like recess 10, can assume and maintain a vertical position totally adjacent to the wall 2c of the shower booth 2, as schematically shown in FIG. 5.

The application means 13 thus substantially act also as anti-turnover means and removable blocking means of the panel 4 with respect to the wall 2c of the shower booth 2.

Advantageously, it is noted that the effect of the application means 13 is such as to allow an easy and fast removal of the panel 4 from the same wall 2c of the shower booth.

According to an embodiment of the present invention, the panel 4 also has a plurality of slits or grids 14, which represent openings for the dissipation of the heat developed by the operation of the lamps 11. The slits are placed in correspondence with the end portions of the box-shaped body 5, which is further equipped with a plurality of valves 15 for the venting of the steam possibly accumulated therein.

According to an embodiment of the present invention, the tanning shower-bath booth 1 is completed by an electric supply cable and by a switch for turning on and off the lamps 11, not shown because conventional.

The operations of turning on and off the lamps 11 can be also performed at a distance, i.e. from inside the shower booth 2, by means of a remote control (suitably equipped with a casing realised with proper material, so as to be water proof), whose signal received by a corresponding sensor of the panel 4.

Advantageously, according to an embodiment of the present invention, the box-shaped body 5 of the panel 4 is realised in light material, e.g. aluminium or plastics, whereas the wall 9 is realised in transparent material, e.g. glass or preferably plastic material commercially known as Perspex.

The application means 13 are conveniently realised in strong and light plastic material, preferably Plexiglas. In a preferred embodiment, schematically shown in FIG. 3, the application means 13 are hooks or staples realised by means of a plastic material layer suitably U-configured.

Now, with particular reference to the example of FIG. 4, a further embodiment of the present invention is described, for which details and co-operating parts having the same structure and operation as the previous embodiment will be indicated with the same reference numbers and abbreviations.

According to this further embodiment of the tanning shower-bath booth, the application means comprise a plurality of pintles 113a, integral with the frame 2d of the shower booth 2, and destined to house and retain in a removable way a respective plurality of straps 113b, fixed to the panel 4. In other words, the plurality of pintles 113a and of straps 113b realises suitable pintle means for said panel 4.

These means, in practice, act both as anti-turnover means of the panel 4 and as hinged means to apply the panel 4 to the shower booth 2.

So, in this way, the movement of the panel 4 is allowed, which can thus assume a position adjacent to the wall 2c of the shower 2 and, if desired, it can be spaced away from the wall, remaining at the same time hinged to the frame 2d thereof, thus ready to be fast and simply replaced in the operating position.

The main advantage of the present invention stays in the possibility of getting “sun” tanned, as effect of the exposition of the body to the irradiation of UV light, and if desired also while having a shower, everything having at disposal a tanning shower-bath booth which, comprising a panel structurally independent from the shower booth, has a low cost and a versatile and simple structure.

Its manoeuvrability in fact allows an easy application and installation, and just as easy removal, thanks to the structurally independent panel, which can thus be easily and simply transported, according to one's needs and desire to different bathrooms and also to different private residences.

Its use versatility is further allowed by the reduced weight of the panel thus realised, by using suitable materials, as previously described.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A shower-bath booth, comprising:
a shower booth having at least a transparent wall,
an UV radiation source associated with the shower booth; and
a panel, structurally independent from the shower booth and equipped with the UV radiation source,
means being provided to apply the panel, in a prefixed spaced relation, to the transparent wall of the shower-bath booth.

2. A shower-bath booth according to claim 1, wherein the panel is removably applied to the transparent wall of the shower-bath booth.

3. A shower-bath booth according to claim 1, wherein the panel is applied outside the transparent wall of the shower-bath booth.

4. A shower-bath booth according to claim 1, wherein the panel is a box-shaped body, defining a space, wherein the UV radiation source is housed.

5. A shower-bath booth according to claim 4, wherein the box-shaped body comprises a wall at least partially transparent closing the space.

6. A shower-bath booth according to claim 1, wherein the means comprise U-shaped elements.

7. A shower-bath booth according to claim 1, wherein the means comprise pintle means.

8. A shower-bath booth according to claim 5, wherein the closing wall of the space is transversally equipped, in correspondence with one end portion thereof, with a step-like recess.

9. A panel comprising:
a box-shaped body defining a space;
an UV radiation source mounted in the space; and
means provided to apply the panel, in a prefixed spaced relation, to a holder.

10. A shower-bath booth, comprising:
a shower booth having at least a transparent wall,
an UV radiation source associated with the shower booth; and
a panel, structurally independent from the shower booth and equipped with the UV radiation source,
means being provided to removably apply the panel, in a prefixed spaced relation, to the transparent wall of the shower-bath booth, outside this transparent wall;
wherein the panel is a box-shaped body, defining a space, wherein the UV radiation source is housed.

11. A shower-bath booth according to claim 10, wherein the box-shaped body comprises a wall at least partially transparent closing the space.

12. A shower-bath booth according to claim 10, wherein the means comprise U-shaped elements.

13. A shower-bath booth according to claim 10, wherein the means comprise pintle means.

14. A shower-bath booth according to claim 10, wherein the closing wall of the space is transversally equipped, in correspondence with one end portion thereof, with a step-like recess.

15. A panel comprising:
a box-shaped body defining a space;
an UV radiation source mounted in the space; and
means provided to apply the panel, in a prefixed spaced relation, to a holder,
wherein box-shaped body comprises a wall at least partially transparent closing the space and the means are chosen between U-shaped elements and pintle means.

16. A shower-bath booth, comprising:
a shower booth having at least a transparent wall,
an UV radiation source associated with the shower booth; and
a panel, structurally independent from the shower booth and equipped with the UV radiation source,
means being provided to apply the panel, in a prefixed spaced relation, to the transparent wall of the shower-bath booth
the panel being a box-shaped body, defining a space, wherein the UV radiation source is housed; the box-shaped body comprising a wall at least partially transparent closing the space.

17. A shower-bath booth according to claim 16, wherein the panel is removably applied to the transparent wall of the shower-bath booth.

18. A shower-bath booth according to claim 16, wherein the panel is applied outside the transparent wall of the shower-bath booth.

19. A shower-bath booth according to claim 1, wherein the means are chosen between U-shaped elements and pintle means.

20. A shower-bath booth according to claim 16, wherein the closing wall of the space is transversally equipped, in correspondence with one end portion thereof, with a step-like recess.
